(19) United States
(12) Patent Application Publication
(10) Pub. No.: US 2015/0352134 A1
(43) Pub. Date: Dec. 10, 2015

(54) COMPOSITION FOR TOPICAL SKIN APPLICATION CONTAINING GINSENOSIDE F2 DERIVED FROM HYDROPONIC GINSENG

(71) Applicant: AMOREPACIFIC CORPORATION, Seoul (KR)

(72) Inventors: Kwon Real RYU, Yongin-si (KR); Dong Hyun KIM, Yongin-si (KR); Ok Chan LEE, Yongin-si (KR); Hyun Hee KIM, Yongin-si (KR); Myeong Hun YEOM, Yongin-si (KR); Jun Cheol CHO, Yongin-si (KR)

(21) Appl. No.: 14/831,294

(22) Filed: Aug. 20, 2015

Related U.S. Application Data
(63) Continuation-in-part of application No. 13/935,801, filed on Jul. 5, 2013, now abandoned.

(30) Foreign Application Priority Data
Jul. 5, 2012 (KR) 10-2012-0073252

(51) Int. Cl. A61K 31/704; A61Q 19/08; A61Q 19/02; A61Q 17/00; A61Q 7/00; A61K 36/258; A61K 8/97; A61K 8/63; A61Q 19/00; A61Q 5/00

(52) U.S. Cl. CPC A61K 31/704; A61Q 19/00; A61Q 19/08; A61Q 19/02; A61Q 5/006; A61Q 7/00; A61Q 19/008; A61K 36/258; A61K 8/97; A61K 8/63; A61Q 17/00; A61Q 17/005; A61K 2800/80

(57) ABSTRACT

Beneficial effects are obtained by topically applying ginsenoside F2 to the skin to improve skin conditions, improve water content on the skin surface and related benefits improving skin problems associated with acne or atopy, providing a skin whitening effect, helping control of sebaceous secretion, tightening skin pores, or improving skin complexion through enhanced blood circulation, but also to improve scalp and hair conditions, such as providing an anti-dandruff effect, promoting hair growth, or preventing generation of grey hair.

This application is a continuation-in-part of U.S. application Ser. No. 13/935,801 filed Jul. 5, 2013 which claims priority to Korean Application No. 10-2012-0073252 filed Jul. 5, 2012, the entire contents of each of which are hereby incorporated by reference.

### BACKGROUND OF THE INVENTION

1. Field of the Invention

The present invention relates to a composition for topical skin application containing ginsenoside F2, and more particularly to a composition containing ginsenoside F2, which is extracted from the roots and leaves of clean fresh ginseng hydroponically grown by an aggregate hydroponic ginseng cultivation system or an aeroponic ginseng cultivation system to yield a higher quantity of ginsenoside F2, where the composition with a high content of ginsenoside F2 can provide beneficial effects not only to improve the entire skin conditions, such as providing an anti-aging effect through excellent antioxidative activity peculiar to ginsenoside F2, improving water content on skin surface, providing an anti-inflammatory effect, improving skin problems associated with acne or atopy, providing a skin whitening effect, helping control of sebaceous secretion, tightening skin pores, or improving skin complexion through enhanced blood circulation, but also to improve scalp and hair conditions, such as providing an anti-dandruff effect, promoting hair growth, or preventing generation of grey hair.

2. Background Art

The human skin acts as a primary protective barrier for the body to protect the internal organs from environmental stimuli, ranging from a change in temperature and humidity to UV radiation or pollutants, and becomes susceptible to changes due to a variety of internal or external factors during aging. The internal factors affecting the skin changes include reduction in the secretion of various hormones produced to control the body's metabolism and deterioration in the functions of immunocytes and the activities of cells, resulting in decreased biosynthesis of immunoproteins essential to the body and other proteins constituting the body. The external factor is ozone depletion that allows an increased amount of ultraviolet rays out of the sun light to reach the Earth and makes the environmental contamination worse to cause an increase in the amount of free radicals and reactive oxygen species, resulting in various skin changes, including not only reduced skin thickness, increased wrinkles, and decreased skin elasticity but also dark skin complexion, frequent occurrence of skin problems, increased occurrence of chloasma, freckle, or senescence spots, sallow skin complexion, and dark pigmented skin.

There have been many attempts to improve skin conditions by adding the existing bioactive substances derived from various animals, plants, or microorganisms to cosmetic products in order to prevent such skin changes caused by the internal or external factors and maintain the skin in healthy conditions.

Thus, the inventors of the present invention have found it out that ginsenoside F2 can provide beneficial effects not only to improve the entire skin conditions, such as providing an anti-aging effect, improving skin wrinkles, providing a skin whitening effect, improving water retention, providing an anti-inflammatory effect, improving acne or other skin problems, improving atopy, improving skin complexion, helping control of sebaceous secretion, or tightening skin pores, but also to improve scalp and hair conditions, such as providing an anti-dandruff effect, promoting hair growth, or preventing generation of grey hair, thereby completing the present invention.

### SUMMARY OF THE INVENTION

It is therefore an object of the present invention to provide a composition for topical skin application containing ginsenoside F2 to improve the entire skin conditions.

To achieve the object of the present invention, there is provided a composition for topical skin application containing, as an active ingredient, ginsenoside F2 extracted from the roots and leaves of clean fresh ginseng cultivated in an aggregate hydroponic ginseng cultivation system or an aeroponic ginseng cultivation system.

The present invention also provides a composition for topical skin application containing ginsenoside F2 as an active ingredient to provide an anti-aging effect.

The present invention also provides a composition for topical skin application containing ginsenoside F2 as an active ingredient to provide a skin whitening effect.

The present invention also provides a composition for topical skin application containing ginsenoside F2 as an active ingredient to provide a skin moisturizing effect.

The present invention also provides a composition for topical skin application containing ginsenoside F2 as an active ingredient to improve acne.

The present invention also provides a composition for topical skin application containing ginsenoside F2 as an active ingredient to improve atopy.

The present invention also provides a composition for topical skin application containing ginsenoside F2 as an active ingredient to improve skin complexion and skin tone.

The present invention also provides a composition for topical skin application containing ginsenoside F2 as an active ingredient to tighten skin pores.

The present invention also provides a composition for topical skin application containing ginsenoside F2 as an active ingredient to control sebaceous secretion.

The present invention also provides a composition for topical skin application containing ginsenoside F2 as an active ingredient to provide an anti-dandruff effect.

The present invention also provides a composition for topical skin application containing ginsenoside F2 as an active ingredient to enhance hair growth.

The present invention also provides a composition for topical skin application containing ginsenoside F2 as an active ingredient to prevent generation of grey hair.

The present invention also provides a composition for topical skin application containing ginsenoside F2 as an active ingredient for use as a natural preservative.

The ginsenoside F2 used in the composition of the present invention is extracted from the roots and leaves of clean fresh ginseng hydroponically grown by an aggregate hydroponic ginseng cultivation system or an aeroponic ginseng cultivation system to yield a higher quantity of ginsenoside F2 than the conventional ginsengs, so the composition can provide beneficial effects not only to improve the entire skin conditions, such as providing an anti-aging effect through excellent antioxidative activity peculiar to ginsenoside F2, improving water content on skin surface, providing an anti-inflammatory effect, improving skin problems associated with acne or atopy, providing a skin whitening effect, helping control of sebaceous secretion, tightening skin pores, or improving skin complexion through enhanced blood circulation, but also to improve scalp and hair conditions, such as providing an anti-dandruff effect, promoting hair growth, or preventing generation of grey hair.

### BRIEF DESCRIPTION OF THE DRAWINGS

FIG. 1 is a graph showing the content of ginsenoside F2 in ginseng leaves as extracted in 80% ethanol.

FIG. 2A shows the analytical results on the ingredients of the leaves from a ginseng grown outdoors and ginsengs hydroponically grown.

FIG. 2B shows the analytical results on the ingredients of the leaves from a ginseng grown outdoors and ginsengs hydroponically grown for 30 days.

FIG. 2C shows the analytical results on the ingredients of the leaves from a ginseng grown outdoors and ginsengs hydroponically grown for 60 days.

FIG. 2D shows the analytical results on the ingredients of the leaves from a ginseng grown outdoors and ginsengs hydroponically grown for 90 days.

FIG. 2E shows the analytical results on the ingredients of the leaves from a ginseng grown outdoors and ginsengs hydroponically grown for 120 days.

FIG. 3 shows hair growth after application of Formulation Example 5 or Comparative Formulation Example 6.

### DETAILED DESCRIPTION OF THE PREFERRED EMBODIMENT

The composition for topical skin application according to the present invention contains ginsenoside F2 as an active ingredient.

The ginsenoside F2 used in the present invention has a structure of the following chemical formula 1:

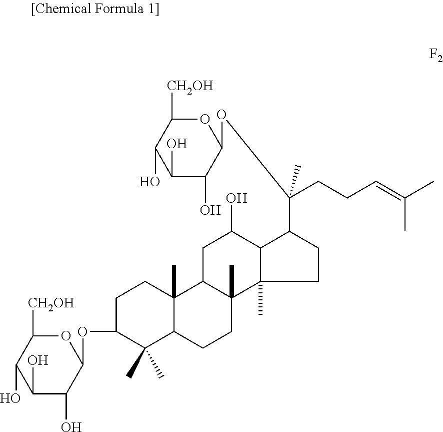

The ginsenoside F2 of the present invention is extracted from the roots or leaves of clean fresh ginseng hydroponically grown and harvested in an aggregate hydroponic ginseng cultivation system or an aeroponic ginseng cultivation system according to the method disclosed in Korean Patent Laid-Open Publication No. 10-2010-0001774.

The method of producing roots and leaves of clean fresh ginseng using the aggregate hydroponic ginseng cultivation system includes the steps of:

a) performing a first acclimatization step of storing a ginseng seedling in a storage greenhouse at 15° C. for one or two days after a release and then tentatively planting the ginseng seedling;

b) performing a second acclimatization step of storing the tentatively planted ginseng seedling in a greenhouse for one or two days to have the ginseng seedling acclimatized in the environment of the greenhouse and then finally planting the ginseng seedling in a mixed medium formed in a bed with a drainage groove;

c) preparing a nutrient solution;

d) supplying an appropriate amount of the nutrient solution for the ginseng seedling; and

e) harvesting after 4 to 5 months.

Further, the method of producing roots and leaves of clean fresh ginseng using the aeroponic ginseng cultivation system includes the steps of:

a) performing a first acclimatization step of storing a ginseng seedling in a storage greenhouse at 15° C. for one or two days after a release and then tentatively planting the ginseng seedling;

b) performing a second acclimatization step of storing the tentatively planted ginseng seedling in a greenhouse for one or two days to have the ginseng seedling acclimatized in the environment of the greenhouse and then finally planting the ginseng seedling in a bed;

c) preparing a nutrient solution;

d) spraying the nutrient solution on the root of the ginseng seedling through a mist nozzle;

e) recycling the used nutrient solution transferred to a nutrient solution tank through a drainage opening formed on the one end of the bed; and

f) harvesting after 4 to 5 months.

The extracts from the roots or leaves of ginseng includes not only the liquid extract obtained by leaching and decoction of the roots or leaves of ginseng grown by the above-mentioned methods but also the concentrate obtained by concentrating the liquid extract in part or the whole, infusions, decoctions, tinctures and fluid extracts prepared by drying the concentrate, chemical substances contained in ginseng to provide principal effects, and the plant itself. Further, the extraction method for the ginsenoside F2 from the ginseng extracts may include any known method.

More specifically, the ginsenoside F2 can be isolated from a ginseng extract prepared with water or an organic solvent according to a method well known in the related art. The organic solvent used in the present invention may include an organic solvent selected from the group consisting of ethanol, methanol, butanol, ether, ethylacetate, chloroform, and a mixture of such an organic solvent and water. The organic solvent is preferably 80% ethanol. Preferably, the extraction temperature is 10 to 80° C., and the extraction time is 3 to 24 hours. The extraction temperature and the extraction time out of the above-defined ranges possibly lead to deterioration in the extraction efficiency or changes of the composition.

The composition of the present invention may contain the ginsenoside F2 at an amount of 0.001 to 50 wt. %, preferably 0.01 to 30 wt. %, more preferably 0.1 to 10 wt. % with respect to the total weight of the composition. The content of the ginsenoside F2 less than 0.001 wt. % provides insignificant efficacy and effects of the ginsenoside F2, whereas the content of the ginsenoside F2 greater than 50 wt. % leads to problems in regards to skin safety or formulation.

As the ginsenoside F2 is an ingredient with high antioxidative activity, the composition of the present invention containing ginsenoside F2 provides a high antioxidant effect and thus can be used as a composition for topical skin application to provide an anti-aging effect, which is excellent in enhancing skin elasticity and improving skin wrinkles

The composition of the present invention can be used as a skin-whitening composition to provide an excellent skin-whitening effect by inhibiting the activity of tyrosinase and suppressing melanogenesis.

The composition of the present invention can be used as a skin-moisturizing composition for topical skin application, which strengthens the skin barrier function and induces the differentiation of keratinocytes on the skin. Therefore, the composition is useful as a composition for topical skin application to prevent and improve xeroderma, atopic dermatitis, contact dermatitis, or psoriasis that is caused by incomplete differentiation of dermal cells.

The composition of the present invention can be used as a composition for topical skin application to improve acne. The composition provides an excellent antibacterial effect, especially to the propionibacterium acne responsible for acne breakouts, and an anti-inflammatory effect.

The composition of the present invention can be used as a composition for topical skin application to improve skin complexion and skin tone. The composition, when applied to the skin, dilates capillary vessels and promotes blood circulation to facilitate the supply of nutrients for the skin and inhibit the skin aging, thereby remarkably improving skin complexion and skin tone.

The composition of the present invention can be used as a composition for topical skin application to tighten skin pores, control sebaceous secretion and improve skin problems. The composition, when applied to the skin, suppresses excessive secretion of sebum, removes reactive oxygen species and promotes synthesis of collagen to tighten skin pores and remarkably prevent skin troubles through a reduction in the expression of inflammatory factors.

The composition of the present invention can be used as an anti-dandruff composition for topical skin application. The composition effectively helps release of toxins accumulated in hair and scalp to clean the scalp and suppresses the proliferation and growth of dandruff bacillus to prevent inflammatory reactions on the scalp. Further, the composition has an excellent anti-oxidative efficacy to inhibit generation and function of reactive oxygen species, so it have effects of relaxing and strengthening the scalp and increasing the intrinsic defensive power of the scalp.

The composition of the present invention can be used as a composition for topical skin application to enhance hair growth. The composition promotes a jump of the hair growth cycle from the telogen (resting) phase to the anagen (growth) phase to accelerate the growth of hair and prevent hair loss.

The composition of the present invention can be used as a composition for topical skin application to prevent generation of grey hair. The composition remarkably increases the expression of MITF in melanocytes to suppress generation of grey hair and promote generation of black hair.

Further, the ginsenoside F2 used in the composition for topical skin application according to the present invention may have an effect as a natural preservative.

The composition for topical skin application according to the present invention may be formulated as a cosmetic composition using a cosmetically or dermatologically acceptable medium or substrate. The composition may be provided in the form of any formulation suitable for topical application. Examples of the formulations for topical application may include solutions, gels, solids, paste anhydrides, oil-in-water emulsions, suspensions, microemulsions, microcapsules, microgranules, ionic (liposome) and/or nonionic vesicular dispersants, cream, skin toner, lotion, powder, ointment, spray, or conceal stick. The composition for topical skin application may also be formulated as a foamed composition or an aerosol composition additionally containing a compressed propellant. The composition of the present invention can be formulated according the methods known in the related art.

Particularly, the composition for topical skin application according to the present invention as used to provide an anti-dandruff function, stimulate hair growth and prevent generation of grey hair can be formulated as a composition for scalp and hair. Examples of the formulation of the composition for scalp and hair may include, but are not specifically limited to, hair tonic, hair nutrition lotion, scalp treatment, hair treatment, shampoo, hair conditioner, hair lotion, or dual purpose treatment for hair and scalp.

Further, the composition of the present invention may contain cosmetically or dermatologically acceptable adjuvants typically used in the cosmetic field, such as lipids, organic solvents, dissolving agents, thickening agents, gelling agents, softening agents, antioxidants, suspending agents, stabilizers, foaming agents, fragrances, surfactants, water, ionic or nonionic emulsifiers, fillers, sequestering agents, chelating agents, preservatives, vitamins, UV-screening agents, humectants, essential oils, dyes, pigments, hydrophilic or lipophilic activators, liposomes, or any other ingredients typically used in cosmetics. The adjuvants are used in an amount generally accepted in the cosmetic or dermatologic field.

Further, the composition of the present invention may contain a skin absorption enhancer in order to increase the skin-improving effects.

Hereinafter, the composition and the effects of the present invention will be described more specifically with reference to experimental examples and formulation examples, which are provided for illustration only to help the understandings of the present invention and not intended to limit the scope of the present invention.

### Reference Example 1

### Hydroponic Cultivation of Ginseng

A ginseng seedling stored at low temperature is stored in a storage greenhouse at 15° C. for 2 days and then tentatively planted for acclimatization in a gardening topsoil (50% coco-peat, 30% peatmoss, 10% vermiculite, and 10% zeolite) containing 70 to 80% water. The temperature in the acclimatization step is maintained in the range of 20 to 23° C. When the new buds appear on the ginseng seedling and turn green after 5 to 7 days, the ginseng seedling is moved to a greenhouse maintained at 25° C. and 80% to 90% in humidity. After 2 days in the greenhouse, the ginseng seedling is finally planted in a bed and supplied with an appropriate amount of a nutrient solution for hydroponic cultivation. The nutrient solution is prepared by mixing macronutrients, such as 6.0 mg/L of NO₃, 0.5 mg/L of NH₄, 4.0 mg/L of K, 2.0 mg/L of Ca, 1.0 mg/L of Mg, 1.5 mg/L of PO₄, and 1.0 mg/L of SO₄, and micronutrients, such as 3.0 mg/L of Fe-EDTA, 0.5 mg/L of Mn, 0.5 mg/L of B, 0.02 mg/L of Cu, 0.05 mg/L of Mo, and 0.05 mg/L of Zn. The pH value is 6.0±0.5. The ion concentration of the nutrient solution is controlled to 0.8±0.1 dS/cm² for 30 days from the development of leaves right after the final planting step to the completion of the acclimatization step, and 1.1±0.1 dS/cm² in the subsequent growth phase.

### Reference Example 2

### Comparison of Ginsenoside F2 Content

The leaves of ginseng normally grown (i.e., grown outdoor) are purchased from Ginseng Retail Center in Geumsan, S. Korea (October in 2011). The leaves of ginseng hydroponically grown are cultivated according to the method of the reference example 1 after final planting in January, 2012, and harvested in 30 days, 60 days, 90 days, and 120 days.

The respective ginseng leaves are dried in a hot air drier at 55° C. for 12 hours to have a same moisture content, finely ground with a mesh (100˜300 mesh) and extracted with 80% ethanol (200 ml per 10 g) for 24 hours. The ginseng leaf extract is made into extract powder through filtration and decompression concentration, dissolved in 80% ethanol to 10,000 ppm and then subjected to HPLC analysis.

The HPLC analysis uses a 2695 separation module and a 2996 PDA detector supplied by Waters Inc. U.S.A. The analytical column is a 250 mm×4.6 mm i.d. Mightysil C18 reverse phase column manufactured by Kanto Chemical, Japan. The mobile phase includes water and acetonitrile. The HPLC analysis is carried out for 85 minutes. More specifically, based on 100% of water and acetonitrile together, the gradient profile progresses from 90% water to 79% water over 0 to 5 minutes, from 79% water to 79% water over 5 to 10 minutes, from 79% water to 77.5% water over 10 to 35 minutes, from 77.5% water to 69% water over 35 to 37 minutes, from 69% water to 50% water over 37 to 77 minutes, from 50% water to 50% water over 77 to 80 minutes, from 50% water to 90% water over 80 to 83 minutes, and from 90% water to 90% water over 83 to 85 minutes.

Further, the standard product of ginsenoside F2 is purchased from AMBO Laboratory, Korea.

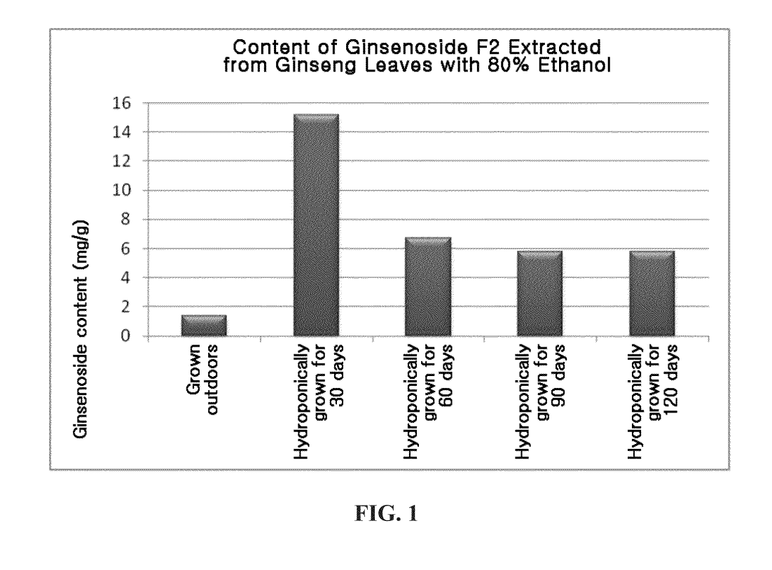

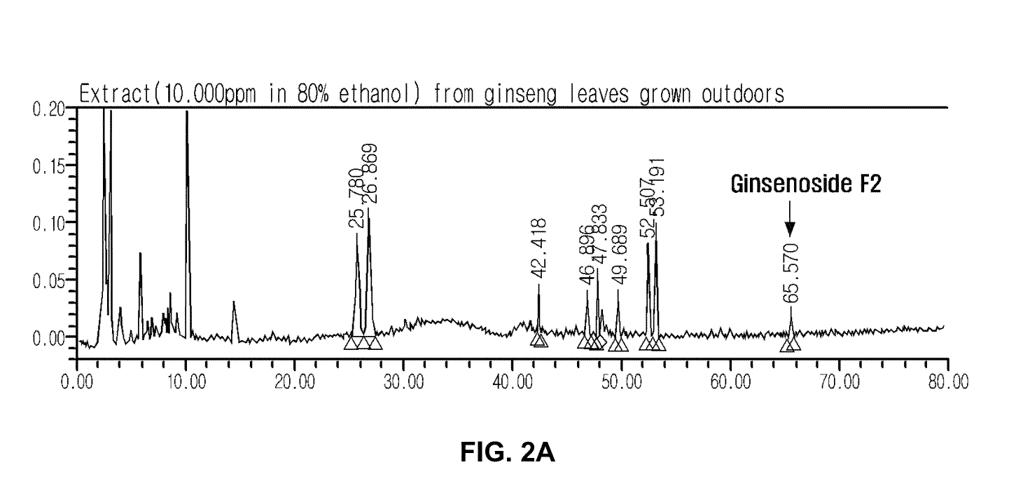

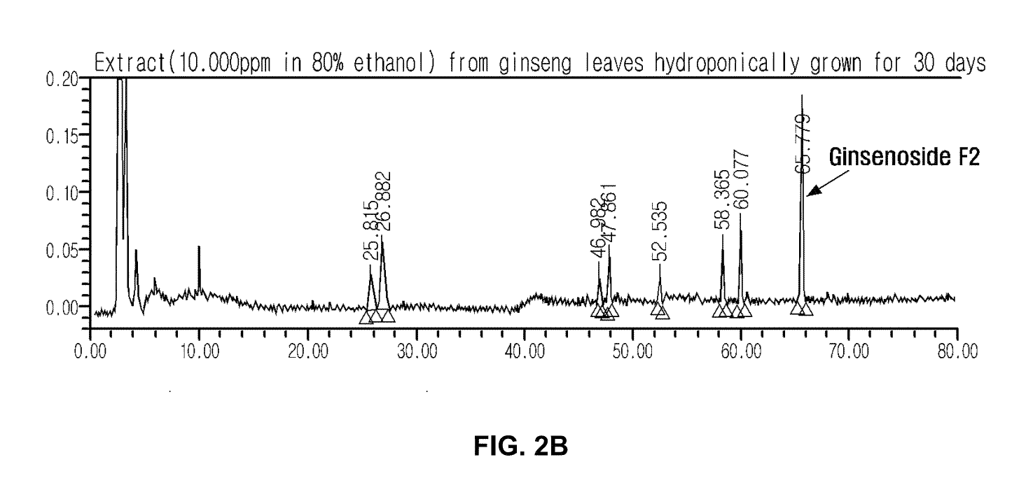

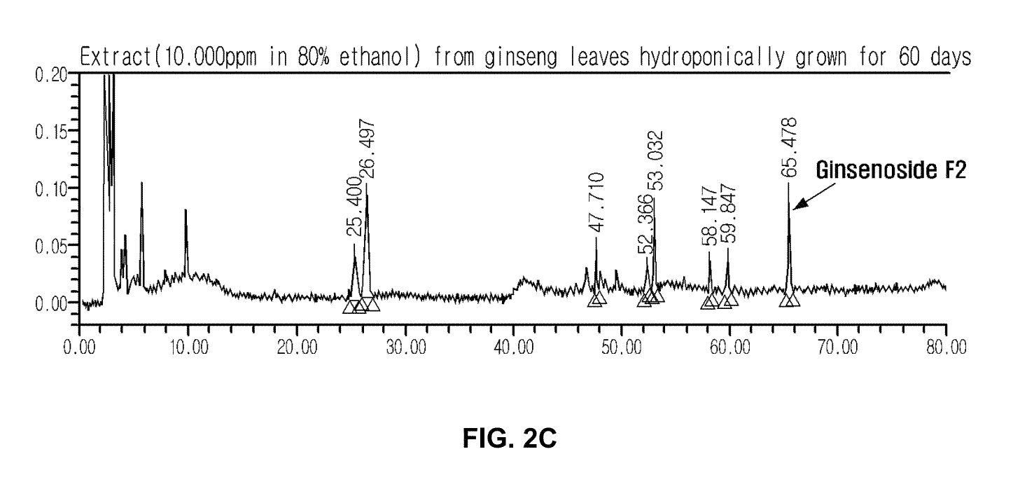

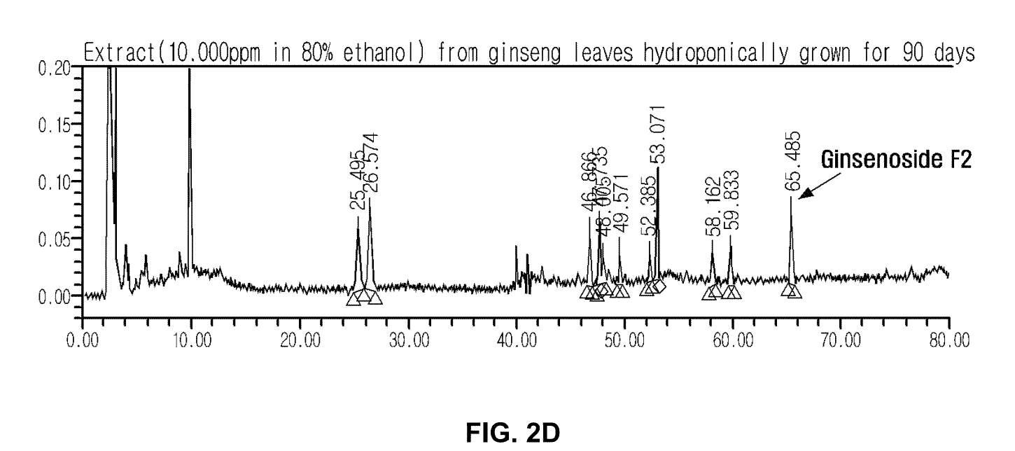

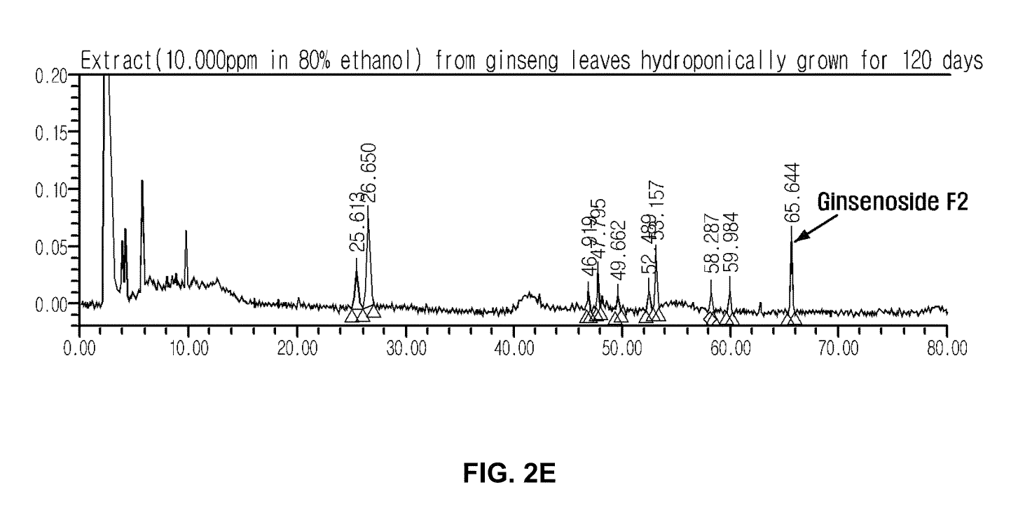

The measurement results are presented in FIGS. 1 and 2. The hydroponically grown ginseng has a considerably high content of ginsenoside F2 relative to the ginseng grown outdoors. Particularly, the ginseng hydroponically grown for 30 days has the highest content of ginsenoside F2, and the ginseng hydroponically grown for more than 30 days has a reduced content of ginsenoside F2.

### Experimental Example 1

### Effect of Inhibiting Formation of Reactive Oxygen Species

5×10⁴ Keratinocytes isolated from the human epidermal tissue are put in each well of a 24-well plate and immobilized for 24 hours. After 16 hours, the keratinocytes are treated with 1% ginsenoside F2. For a comparison, the control group is not treated with ginsenoside F2. Each well is removed of the culture medium after 2 hours and then fed with 100 μl of a phosphate-buffered saline (PBS) solution. The keratinocytes are exposed to 30 mJ/cm² of ultraviolet radiation under an ultraviolet B (UVB) lamp (Model: F15T8, UV B 15 W, Sankyo Denki Co., Ltd., Japan). Each well is removed of the PBS solution and then supplied with 200 μl of the keratinocyte culture medium. The well is treated with ginsenoside F2 again. Then, the quantity of the reactive oxygen species (ROS) increased by the UV stimulation is determined at defined time intervals. The quantity of the reactive oxygen species (ROS) is determined according to the method that includes measuring the fluorescence of dichlorofluorescin diacetate (DCF-DA) (Tan et al., 1998, J. Cell Biol. Vol. 141, pp 1423-1432). The calculation results of the ratio to the ROS of the control group treated with vehicles alone are presented in Table 1.

TABLE 1

| | Elapsed time after exposure | | |
|---|---|---|---|
| | to 30 mJ/cm² of UVB | | |
| Test substance | 0 hr | 2 hrs | 3 hrs |
| Vehicle | 100 | 244 | 287 |
| UVB + vehicle | 100 | 325 | 381 |
| UVB + | 100 | 302 | 354 |
| ginsenoside Rb1 | | | |
| UVB + | 100 | 251 | 286 |
| ginsenoside F2 | | | |

As can be seen from the results of Table 1, the ginsenoside F2 of the present invention effectively suppresses the generation of reactive oxygen species (ROS) known to cause damages on the skin cells under UV radiation and has such an outstanding antioxidative efficacy as to inhibit the generation of reactive oxygen species (ROS) after exposure to UV radiation to the almost equivalent level to the case that the skin cells are not exposed to UV radiation. It is therefore revealed that the ginsenoside F2 of the present invention suppresses oxidation and aging to keep skin pores from getting widened and inhibits breakouts of skin irritations to improve skin problems.

### Experimental Example 2

### Measurement of Elastase Inhibitory Effect

The elastase inhibitory function of the ginsenoside F2 is measured in comparison with that of EGCG. The elastase and the substrate used herein are purchased from Sigma-Aldrich, U.S.A. (Cat. No. E0127).

The elastase inhibitory function is measured according to the following method.

In a 96-well plate, ginsenoside F2 (200 μL) and 50 μL of a 20 μg/mL elastase type III solution are mixed with a 10 mg/L tris-HCL buffer solution (pH 8.0). The positive control is EGCG 250 μM, and the negative control which is a non-treated group is distilled water. Then, 100 μL of 0.4514 mg/mL N-succinyl-ala-ala-ala-p-nitroanilide prepared with the buffer solution is added to the mixture and allowed to react at 25° C. for 15 minutes. After completion of the reaction, the absorbance at 415 nm was measured. The blank test is performed in the same manner as described above to achieve calibration verification.

The elastase inhibitory function is calculated according to the following mathematical formula 1, and the results are presented in Table 2.

Elastase inhibition rate (%)={1−(C−D)/(A−B)}×100 [Mathematical Formula 1]

A: Absorbance at 415 nm with enzyme and no test substance.

B: Absorbance at 415 nm with no test substance and no enzyme.

C: Absorbance at 415 nm with test substance and enzyme.

D: Absorbance at 415 nm with test substance and no enzyme.

TABLE 2

| Compound | Inhibition rate (%) |
|---|---|
| Control (not treated) | 0 |
| EGCG | 65 |
| Ginsenoside Rb1 | 71 |
| Ginsenoside F2 | 78 |

As can be seen from Table 2, the ginsenoside F2 shows the far higher level of the elastase inhibition rate than EGCG which is known as an existing elastase inhibitor. Accordingly, the ginsenoside F2 of the present invention has an excellent elastase inhibitory effect.

### Experimental Example 3

### Collagenase (MMP-1) Inhibitory Effect

The ginsenoside F2 of the present invention is measured in regards to the inhibitory effect on the generation of collagenase in comparison with retinoic acid.

Human fibroblasts are put in each well of a 96-well microtiter plate filled with a Dulbecco's modified eagle's medium (DMEM) containing 2.5% fetal bovine serum at a rate of 5,000 fibroblasts per well and cultures in an incubator at 37° C. in the atmosphere containing 5% CO₂ until they grow by about 70 to 80%. The human fibroblasts are treated with ginsenoside F2 or retinoic acid at a concentration of 10 μg/mL for 24 hours. Then, the cell culture medium is collected.

The production yield of collagenase in the collected cell culture medium is measured using a collagenase measurement instrument commercially available (Catalog #. RPN 2610, USA Amersham Pharma Inc.). Firstly, the collected cell culture medium is put in each well of a 96-well plate uniformly coated with the primary collagenase antibodies and then subjected to antigen-antibody reactions in a thermostat container for 3 hours. After 3 hours, the secondary collagenase antibodies coupled to the chromophore groups are put in each well of the 96-well plate and then allowed to react for more 15 minutes. After 15 minutes, a color-causing substance (3,3′,5,5′-tetramethylbenzidine, Sigma) is put in each well of the 96-well plate to cause coloration at the room temperature for 15 minutes, and then 1M sulfuric acid is added to terminate the coloration reaction so that the reaction mixture becomes yellow. The intensity of yellowness in the reaction mixture is varied depending on the progress of the reaction.

The absorbance of the yellow-colored 96-well plate is measured at 405 nm with a spectrophotometer, and the expression level of collagenase is calculated according to the following mathematical formula 2. The results are presented in Table 3. In this regard, the absorbance of the cell culture medium collected from the group not treated with the composition of the present invention is considered as the absorbance of the control.

$$Collagenase expression level ( \% ) = \frac{Absorbance of test cell group}{Absorbance of control} \times 100 \qquad [ Mathematical Formula 2 ]$$

TABLE 3

| Compound | Collagenase expression level(%) |
|---|---|
| Control (not treated) | 100 |
| Retinoic acid | 75 |
| Ginsenoside Rb1 | 74 |
| Ginsenoside F2 | 73 |

As can be seen from Table 3, the ginsenoside F2 has an equivalent collagenase expression inhibitory effect to retinoic acid which is known as a conventional collagenase expression inhibitor.

From the experimental results, it is concluded that the ginsenoside F2 of the present invention has an effect of inhibiting matrix metalloproteinase-1 (MMP-1) activity.

### Formulation Example 1 and Comparative Formulation Example 1

A nutrition cream is prepared by a typical method according to the composition of the following Table 4 (unit: wt. %).

TABLE 4

| | | Compar- | Compar- |
|---|---|---|---|
| | | ative | ative |
| | Formu- | Formu- | Formu- |
| | lation | lation | lation |
| | Example | Example | Example |
| Ingredients | 1 | 1 | 1-1 |
| Purified water | Up to 100 | Up to 100 | Up to 100 |
| Ginsenoside F2 | 0.1 | — | — |
| Ginsenoside Rb1 | — | — | 0.1 |
| Hydrogenated | 1.50 | 1.50 | 1.50 |
| vegetable oil | | | |
| Stearic acid | 0.60 | 0.60 | 0.60 |
| Glycerol stearate | 1.00 | 1.00 | 1.00 |
| Stearyl alcohol | 2.00 | 2.00 | 2.00 |
| polyglyceryl-10 | 1.00 | 1.00 | 1.00 |
| penta- | | | |
| stearate&behenyl | | | |
| alcohol& sodium | | | |
| stearoyl lactylate | | | |
| Arachidyl behenyl | 1.00 | 1.00 | 1.00 |
| alcohol& arachidyl | | | |
| glucoside | | | |
| Cetyl aryl | 2.00 | 2.00 | 2.00 |
| alcohol&cetearyl | | | |
| glucoside | | | |
| PEG-100 | 1.50 | 1.50 | 1.50 |
| stearate&glycerol | | | |
| oleate&propylene | | | |
| glycol | | | |
| Caprylic/capric | 11.00 | 11.00 | 11.00 |
| triglyceride | | | |
| Cyclomethicone | 6.00 | 6.00 | 6.00 |
| Preservative, | q.s. | q.s. | q.s. |
| fragrance | | | |
| Triethanol amine | 0.1 | 0.1 | 0.1 |

### Experimental Example 4

### Evaluation of Skin Elasticity Improvement

The nutrition creams of the formulation example 1 and the comparative formulation example 1 are used in order to evaluate the composition of the present invention in regards to the effect of enhancing skin elasticity in human.

20 healthy female adults in their 30's or 40's are divided into two groups. The one group includes 10 female adults treated on the face with the nutrition cream prepared in the formulation example 1 once a day for 12 weeks, while the other group includes 10 female adults treated on the face with the nutrition cream prepared in the comparative formulation example 1 once a day for 12 weeks. Then, the skin elasticity for each object is measured with a skin elasticity measurer (Cutometer® SEM 575, C+K Electronic Co., Germany). The results are presented in Table 5. The measurement values of Table 5 are the value of ΔR8 of Cutometer® SEM 575, where ΔR8 represents the skin viscoelasticity.

TABLE 5

| Test product | Skin elasticity effect |
|---|---|
| Formulation example 1 | 0.32 |
| Comparative formulation | 0.10 |
| example 1 | |
| Comparative formulation | 0.19 |
| example 1-1 | |

As can be seen from Table 5, the test group treated with the formulation example 1 containing ginsenoside F2 according to the present invention is more enhanced in the skin elasticity than the test group treated with the comparative formulation example 1.

It is therefore concluded that the composition containing ginsenoside F2 according to the present invention is very effective in enhancing the skin elasticity.

### Experimental Example 5

### Evaluation of Skin Wrinkle Improvement

The nutrition creams of the formulation example 1 and the comparative formulation example 1 are used in order to evaluate the composition of the present invention in regards to the effect of improving skin wrinkles in human.

The procedures are performed in the manner as described below to evaluate the skin wrinkle improving effect of the formulation example 1 and the comparative formulation example 1. 20 healthy female adults in their 40's are divided into two groups. The one group includes 10 female adults treated on the face with the nutrition cream of the formulation example 1 once a day for 12 weeks, while the other group includes 10 female adults treated on the face with the nutrition cream of the comparative formulation example 1 once a day for 12 weeks. Then, a replica is made out of silicone to evaluate the severity of wrinkling on the skin with a skin visiometer (Visiometer®, SV600, Courage+Khazaka electronic GmbH, Germany) and do an image analysis. The results are presented in Table 6. The values in Table 6 are an average value of the respective parameters 12 weeks after an application subtracted by the variable before the application.

TABLE 6

| Clinical results after | | | | | |
|---|---|---|---|---|---|
| 12-week use | R1 | R2 | R3 | R4 | R5 |
| Formulation example 1 | 0.13 | 0.12 | 0.09 | 0.01 | 0.01 |
| Comparative formulation | 0.27 | 0.26 | 0.21 | 0.03 | 0.03 |
| example 1 | | | | | |
| Comparative formulation | 0.21 | 0.18 | 0.15 | 0.03 | 0.02 |
| example 1-1 | | | | | |

R1: Difference between the highest and lowest values of skin wrinkle contour.
R2: The average of R1s for the skin wrinkle contour divided into five portions.
R3: The highest one of the five R1's.
R4: The average of the values obtained by subtracting the respective crests and troughs from the baseline of the wrinkle contour.
R5: The value obtained by subtracting each wrinkle outline from the baseline of the wrinkle contour.

As can be seen from Table 6, the composition for topical skin application according to the formulation example 1 has a remarkably excellent effect of improving skin wrinkles

### Experimental Example 6

### Tyrosinase Inhibitory Effect

The tyrosinase as used herein is an enzyme extracted from mushrooms and purchased from Sigma-Aldrich. Firstly, tyrosine as a substrate is dissolved in distilled water to prepare a 0.3 mg/mL solution, which is added dropwise to a test tube at an amount of 1.0 mL each time. To the solution are added 1.0 mL of a potassium phosphate buffer solution (0.1 mol, pH 6.8) and 0.7 mL of distilled water.

The ginsenoside F2 of the present invention is mixed with an ethanol solution at an appropriate concentration to prepare a sample solution. 0.2 mL of the sample solution is added to the reactant solution and then allowed to have a reaction in a thermostat container at 37° C. for 10 minutes. In this regards, the control is the reactant solution containing 0.2 mL of a solvent alone in place of each sample solution. The positive control uses ascorbic acid. 0.1 mL of a 2500 unit/mL tyrosinase solution is added to the reactant solution, which is then allowed to have a reaction again in a thermostat container at 37° C. for 10 minutes. The test tube filled with the reactant solution is put into an ice water to cause quenching and thus stop the reaction. The absorbance at 475 nm is measured with a photoelectric spectrophotometer. The measurement results are presented in Table 7. The tyrosinase inhibitory effect is calculated according to the following mathematical formula 3.

$$Tyrosinase Inhibitory Rate ( \% ) = 100 - ( \frac{Absorbance of test substance}{Absorbance of control} \times 100 ) \qquad [ Mathematical Formula 3 ]$$

TABLE 7

| Test substance | Tyrosinase inhibition rate (%) |
|---|---|
| Control (not treated) | 0 |
| Ascorbic acid | 52 |
| Ginsenoside Rb1 | 60 |
| Ginsenoside F2 | 69 |

As can be seen from Table 7, the ginsenoside F2 of the present invention has a far higher level of the tyrosinase inhibition rate than ascorbic acid which is a known tyrosinase inhibitor, thereby providing an excellent skin whitening effect.

### Experimental Example 7

### Melanogenesis Inhibitory Effect Using B16/F10 Melanoma Cells

As a test substance, a sample containing 0.001 wt. % of ginsenoside F2 or kojic acid is added to a culture medium of B16/F10 melanoma cells (available from Korean Cell Line Bank) at a predetermined concentration. The culture medium is eliminated after 3-day cultivation. The cells are washed with PBS and dissolved in 1N NaOH. Then, the absorbance at 405 nm is measured. The control is the cells not treated with any test substance. In comparison with the melanin content in the control group, the inhibitory ability of each test substance against melanogenesis is measured. The melanogenesis inhibition rate is determined according to the following mathematical formula 4. The results are presented in Table 8.

$$Melanogenesis Inhibitory Rate ( \% ) = 100 - ( \frac{Absorbance of test substance}{Absorbance of control} \times 100 ) \qquad [ Mathematical Formula 4 ]$$

TABLE 8

| Test substance | Melanogenesis inhibition rate (%) |
|---|---|
| Control (not treated) | 0 |
| Kojic acid | 53 |
| Ginsenoside Rb1 | 61 |
| Ginsenoside F2 | 72 |

As can be seen from Table 8, the ginsenoside F2 of the present invention has a far higher level of the melanogenesis inhibition rate than kojic acid which is a known melanogenesis inhibitor, thereby providing an excellent skin whitening effect.

### Experimental Example 8

### Effect of Increasing Water Content in Skin

The nutrition creams prepared in the formulation example 1 and the comparative formulation example 1 are used to evaluate the ginsenoside F2 in regards to the effect of increasing water content in the skin. The evaluations are performed as follows.

20 dry-skinned male or female adults in their 40's or 50's are divided into two groups. The one group includes 10 adults treated on the face with the nutrition cream prepared in the formulation example 1 twice a day for 4 weeks, while the other group includes 10 adults treated on the face with the nutrition cream prepared in the comparative formulation example 1 twice a day for 4 weeks. Then, the water content in the skin is measured with a skin water content measurer (Corneometer® CM825, C+K Electronic Co., Germany) under constant-temperature and constant-humidity conditions (24° C., RH 40%) before the initial application, one week, two weeks and four weeks after the application, and two weeks after termination of the application. The results are presented in Table 9. The measurement values of Table 9 are the percentage of the increment of the measurement value after a predetermined period of treatment with the water content measured right before the start of the testing.

TABLE 9

| | Water content increment (%) | | | |
|---|---|---|---|---|
| | After 1 | After 2 | After 4 | After 6 |
| Test group | week | weeks | weeks | weeks |
| Formulation example 1 | 31 | 33 | 34 | 33 |
| Comparative formulation | 30 | 32 | 32 | 15 |
| example 1 | | | | |
| Comparative formulation | 31 | 31 | 33 | 26 |
| example 1-1 | | | | |

As can be seen from Table 9, the topical application of the comparative formulation example 1 increases the water content in the skin by about 30% until 4 weeks of the treatment but decreases the water content in the skin after termination of the treatment. Contrarily, the topical application of the formulation example 1 containing ginsenoside F2 results in an continuous increase of the water content in the skin by at least about 30% even after termination of the treatment. This shows that the composition of the present invention containing ginsenoside F2 has an excellent skin moisturizing effect.

### Experimental Example 9

### Effect of Promoting Differentiation of Keratinocytes

The quantity of CE (Cornified Envelop) generated during differentiation of keratinocytes is measured in terms of absorbance in order to evaluate the ginsenoside F2 in regards to the effect of promoting differentiation of keratinocytes.

Human keratinocytes isolated from the epidermis of a newborn baby and subjected to a primary cultivation are immobilized in the bottom of a cultivation flask, treated with ginsenoside F2 applied to the culture medium to a concentration of 5 ppm, and cultured for 5 days until the keratinocytes grow to take up about 70 to 80% of the bottom area. In this regard, the negative control is the group treated with low calcium (0.03 mM), while the positive control is the group treated with high calcium (1.2 mM). Then, the cultured keratinocytes are harvested, washed with phosphate-buffered saline (PBS), treated with a 10 mM tris-HCl buffer solution (pH 7.4) containing 2% sodium dodecyl sulfate (SDS) and 20 mM dithiothreitol (DTT), and then subjected to sonication, boiling, and centrifugal isolation. The precipitation is suspended in 1 mL of PBS to measure the absorbance at 340 nm. Apart from this process, a part of the solution after sonication is collected and measured in regards to the protein content, which is determined as a reference in evaluating the degree of differentiation. The results are presented in Table 10.

TABLE 10

| Test substance | Differentiation in keratinocytes (%) |
|---|---|
| Low calcium (0.03 mM) solution | 100 |
| (negative control) | |
| High calcium (1.2 mM) solution | 210 |
| (positive control) | |
| Ginsenoside Rb1 | 265 |
| Ginsenoside F2 | 309 |

As can be seen from Table 10, the ginsenoside F2 has an excellent effect to promote differentiation of keratinocytes.

### Experimental Example 10

### Effect of Recovering Skin Barrier Function

The procedures are performed as follows to measure the effect of the ginsenoside F2 on the recovery of the skin barrier function deteriorated by the skin damage. 10 male or female adults are subjected to damage on the skin barrier function by using the tape stripping method on the upper arm. Then, the formulation example 2 and the comparative formulation example 2 prepared according to Table 11 are respectively applied to the upper arms once a day for 7 days to measure the recovery from transepidermal water loss (TEWL) with a Vapometer® (Delfin, Finland). In this regard, the comparative formulation example 2 is vehicle as the negative control. The results are presented in Table 12. The results of Table 12 are compared with respect to 100% for the difference before and after the skin barrier damage.

TABLE 11

| | | Comparative | Comparative |
|---|---|---|---|
| | Formulation | formulation | formulation |
| Ingredient | example 2 | example 2 | example 2-1 |
| Distilled water | 69 | 70 | 69 |
| Propylene glycol | 30 | 30 | 30 |
| Ginsenoside Rb1 | — | — | 1 |
| Ginsenoside F2 | 1 | — | — |

TABLE 12

| | | | Comparative | Comparative |
|---|---|---|---|---|
| | | Formulation | formulation | formulation |
| Test group | | example 2 | example 2 | example 2-1 |
| TWEL | Before application | 100 | 100 | 100 |
| change | 1 day | 119.8 | 121.4 | 121.1 |
| (%) | 2 days | 120.9 | 112.7 | 116.4 |
| | 3 days | 118.4 | 98.3 | 110.1 |
| | 4 days | 117.2 | 70.5 | 79.8 |
| | 5 days | 111.7 | 62.3 | 71.2 |
| | 6 days | 106.5 | 43.5 | 56.4 |

As can be seen from Table 12, the treatment with the comparative formulation example 2 not containing ginsenoside F2 leads to an increase in the transepidermal water loss with an elapse of time, while the treatment with the formulation example 2 containing ginsenoside F2 brings about a rapid recovery from the transepidermal water loss to recover the skin barrier damage.

### Experimental Example 11

### Effect of Improving Skin Complexion

The blood circulation in the skin is measured with a laser Doppler perfusion imager (LDPI) in order to evaluate the cosmetic composition of the present invention in regards to the effect of promoting blood circulation in the skin. The LDPI is an instrument widely used as a device to measure the blood circulation in the skin that is highly sensitive enough to measure the blood flow in arteriols and venules as well as the circulation rate and quantity of the blood in the capillary vessels of the skin.

After water wash on the face with a soap and adaptation for 30 minutes in a constant-temperature, constant-humidity room, the initial value is measured with the LDPI. Firstly, 30 female adults normally with cold hands and feet are subjected to the LDPI measurement to measure the initial blood flow at the portion below the forehead. Then, the female objects are treated with the formulation example 1 and the comparative formulation example 1 for one week and evaluated in regards to the blood flow after the treatment. The measurements of the blood flow and the initial measurement are compared to determine the change of the blood flow in the skin. The results are presented in Table 13.

TABLE 13
LDPI measurements (skin blood flow) before and after application
of cosmetics

| | Change of skin blood flow (%) |
|---|---|
| Test substance | after one-week application |
| Formulation example 1 | 13 |
| Comparative formulation example 1 | 5 |
| Comparative formulation example 1-1 | 10 |

As can be seen from the results of Table 13, the cosmetic composition of the present invention remarkably increases the blood flow in the skin to improve the skin complexion through promotion of blood circulation, with respect to the comparative formulation example 1 not containing ginsenoside F2. This implicitly shows that the cosmetic composition containing ginsenoside F2 according to the present invention effectively transfers nutrients to the skin and contributes to the suppression and delay of the skin aging.

### Experimental Example 12

### Effect of Improving Skin Tone

In order to evaluate the formulation example 1 and the comparative formulation example 1 in regards to the effect of improving skin tone, 30 objects are treated independently with the formulation example 1 and the comparative formulation example 1 (once a day in the evening for one week) and then evaluated in regards to the skin tone improvement by way of Facial Stage® DM-3 equipment (Moritex, Japan). The skin tone improvement is determined by the change of the skin brightness and color measurements. The results are presented in Table 14. The greater change of the skin brightness and color shows the higher level of the skin tone improvement.

TABLE 14

| | Skin tone improvement (%) | |
|---|---|---|
| | Brightness (average ± | Color (average ± |
| Test substance | standard derivative) | standard derivative) |
| Formulation example 1 | 15 ± 3.24 | 12 ± 2.34 |
| Comparative formulation | 5 ± 2.34 | 5 ± 2.05 |
| example 1 | | |
| Comparative formulation | 11 ± 3.41 | 8.2 ± 2.17 |
| example 1-1 | | |

As can be seen from Table 14, the comparative formulation example 1 not containing the ginsenoside F2 of the present invention does not show a significant effect of improving the skin tone, while the formulation example 1 containing ginsenoside F2 as an active ingredient greatly improves the skin tone after its application.

### Experimental Example 13

### Effect of Tightening Skin Pores

1. Effect of Tightening Skin Pores by Promotion of Collagen Biosynthesis

The ginsenoside F2 of the present invention is compared with TGF-β in regards to the effect of promoting biosynthesis of collagen. Firstly, fibroblasts are seeded in 24 wells at a rate of 10⁵ per well and cultured until the growth reaches 90%. The fibroblasts are cultured in a non-serum culture medium for 24 hours, treated with each 10 ng/ml of ginsenoside F2 or TGF-β dissolved in the non-serum culture medium, and then cultured in a CO₂ incubator for 24 hours. The supernatant liquid is collected to determine an increase or decrease of procollagen using a procollagen type (I) ELISA kit. The results are presented in Table 15. The collagen synthesis ability is denoted with respect to 100% for the non-treated group.

TABLE 15

| Test substance | Collagen synthesis ability (%) |
|---|---|
| Control group (not treated) | 100 |
| TGF-β | 183.5 |
| Ginsenoside Rb1 | 188.3 |
| Ginsenoside F2 | 191.6 |

As can be seen from Table 15, the ginsenoside F2 of the present invention has a higher level of collagen synthesis ability than the positive control, TGF-β. This shows that the ginsenoside F2 of the present invention increases the production of collagen around the skin pores to tighten the wide skin pores.

2. The Effect of Tightening Skin Pores

The procedures are performed as follows to evaluate the formulation example 1 and the comparative formulation example 1 in regards to the effect of tightening skin pores. 20 male or female adults with wide skin pores are divided into two groups. The one group includes 10 objects treated on the face with the nutrition cream prepared in the formulation example 1 daily for 4 weeks, while the other group includes 10 objects treated on the face with the nutrition cream prepared in the comparative formulation example 1 daily for 4 weeks. To evaluate the effect of tightening skin pores, pictures of the skin taken before the testing and 4 weeks after the testing are shown to the experts for visual evaluation. The results are presented in Table 16, where grade 0 denotes no tightening effect on the skin pores; and grade 5 denotes the highest effect of tightening the skin pores.

TABLE 16

| Test substance | Evaluation grade |
|---|---|
| Formulation example 1 | 4 |
| Comparative formulation example 1 | 0 |
| Comparative formulation example 1-1 | 2 |

As can be seen from Table 16, the comparative formulation example 1 has no effect of tightening skin pores, while the formulation example 1 has a skin pore tightening effect visually recognizable. This implicitly shows that the ginsenoside F2 of the present invention has an excellent effect of reducing the size of the skin pores.

### Experimental Example 14

### Inhibitory Effect on Sebaceous Secretion

1. Inhibitory Effect on Excessive Sebaceous Secretion by 5α-Reductase Activity Inhibition

In order to evaluate the inhibitory effect on the activity of 5α-reductase, the conversion rate from [¹⁴C]testosterone to [¹⁴C]dihydrotestosterone (DHT) in HEK293-5α R2 cells is measured. P3×FLAG-CMV-5α R2 is transduced into HEK293 cells, which are cultured in a 24-well plate at a rate of 2.5×10⁵ cells per well (Park et al., 2003, JDS. Vol. 31, pp. 191-98). The next day, the used culture medium is replaced with a new culture medium containing an enzyme substrate and an inhibitor. The substrate of the culture medium is 0.05 μCi [¹⁴C]testosterone (Amersham Pharmacia Biotech, UK).

To evaluate the degree of 5α-reductase activity inhibition, the cells are treated with ginsenoside F2 and then cultured in a 5% CO₂ incubator at 37° C. for 2 hours. In this regard, the negative control is the group not treated with ginsenoside F2, while the positive control is the group treated with finasteride. Subsequently, the culture medium is collected to extract steroids with 800 μl of ethylacetate, and the supernatant organic solvent phase is isolated and dried. The residue is dissolved in 50 μl of ethylacetate and developed on silica plastic sheet kieselgel 60 F254 using an ethylacetate-hexane (1:1) solvent.

The plastic sheet is dried out in the air and measured in regards to the abundance of isotope using a bath system. The dry plastic sheet together with an X-ray film is put in a bath cassette. After one week, the abundance of isotope of testosterone and dihydrotestosterone remaining on the film is measured to calculate the conversion rate and the inhibition rate according to the following mathematical formulas 5 and 6, respectively. The results are presented in Table 17.

$$\begin{matrix}Conversion rate ( \% ) = \frac{Radioactivity at DHT region}{Total radioactivity} \times 100 & [ Mathematical Formula 5 ] \\ Inhibition rate ( \% ) = \frac{( Conversion rate of control - Conversion rate of test substance )}{Conversion rate of control} \times 100 & [ Mathematical Formula 6 ]\end{matrix}$$

TABLE 17

| Test substance | Conversion rate (%) | Inhibition rate (%) |
|---|---|---|
| Negative control | 48.0 | — |
| Positive control | 27.6 | 42.5 |
| Ginsenoside F2 | 15.4 | 59.7 |

As can be seen from Table 17, the ginsenoside F2 can interrupt conversion of testosterone to dihydrotestosterone by effectively inhibiting the activity of 5α-reductase enzyme responsible for conversion of testosterone to dihydrotestosterone which binds to cytoplasmic receptor proteins and enters the nuclear to activate sebaceous gland cells to promote the differentiation of the sebaceous gland cells and thus cause excessive sebaceous secretions. Further, the ginsenoside F2 has a more powerful inhibitory effect on the activity of 5α-reductase enzyme than finasteride known as a conventional 5α-reductase inhibitor. It is therefore revealed that the ginsenoside F2 of the present invention can suppress excessive sebaceous secretions by effectively inhibiting the activity of 5α-reductase enzyme.

2 Inhibitory Effect on Sebaceous Secretions

The procedures are performed as follows to evaluate the formulation example 1 and the comparative formulation example 1 in regards to the inhibitory effect on sebaceous secretions. 30 male or female objects with excessive sebaceous secretions are told to apply the nutrition cream of the formulation example 1 or the comparative formulation example 1 on a defined region of the skin daily for 4 weeks. To evaluate the effect of reducing sebaceous secretions, a sebaceous secretion measurer (Sebumeter® SM810, C+K Electronic Co., Germany) is used to determine the average decrement (%) of sebaceous secretions in 2 weeks and 4 weeks, respectively. The results are presented in Table 18.

TABLE 18

| | Sebaceous secretion decrement (%) | |
|---|---|---|
| Test substance | After 2 weeks | After 4 weeks |
| Formulation example 1 | 44 | 49 |
| Comparative formulation example 1 | 5 | 5 |

As can be seen from Table 18, the formulation example 1 containing the ginsenoside F2 of the present invention as an active ingredient can more effectively suppress excessive sebaceous secretions than the comparative formulation example 1 not containing the ginsenoside F2.

### Formulation Example 3 and Comparative Formulation Examples 3 and 4

The formulation example 3 and the comparative formulation examples 3 and 4 are prepared according to the composition (components and contents (wt. %)) given in Table 19. More specifically, the formulation example 3 contains ginsenoside F2. The comparative formulation example 3 does not contain an active ingredient for improvement of the acne skin. The comparative formulation example 4 contains a standard substance as a reference for antibacterial activity, that is, erythromycin used as an acne treatment.

The preparation methods for the formulation example 3 and the comparative formulation examples 3 and 4 are as follows. The ingredients in phase A of Table 19 are completely dissolved, and the ingredients in phase B are completely dissolved in a separate dissolution tank. The phase B is added to the phase A and made miscible with the phase A. The ingredients in phase C are added to the mixture according to the mixing ratios of Table 19. The resulting mixture is homogenized and then filtered to prepare the compositions of the present invention.

TABLE 19

| | | | Compar- | Compar- | Compar- |
|---|---|---|---|---|---|
| | | | ative | ative | ative |
| | | | formu- | formu- | formu- |
| | | | lation | lation | lation |
| | | Formulation | example | example | example |
| Div. | | example 3 | 3 | 4 | 3-1 |
| A | Deionized Water | Up to 100 | Up to 100 | Up to 100 | Up to 100 |
| | EDTA-2Na | 0.02 | 0.02 | 0.02 | 0.02 |
| | Glycerin | 5.0 | 5.0 | 5.0 | 5.0 |
| | Ethanol | 2.0 | 2.0 | 2.0 | 2.0 |
| B | PEG-60 | | | | |
| | hydrogenated | 0.4 | 0.4 | 0.4 | 0.4 |
| | castor oil | | | | |
| | Perfume | 0.04 | 0.04 | 0.04 | 0.04 |
| C | Ginsenoside F2 | 5.0 | — | — | — |
| | Ginsenoside Rb1 | — | — | — | 5.0 |
| | Erythromycin | — | — | 5.0 | — |

### Experimental Example 15

### Test of Antibacterial Ability to Propionibacterium Acne

The cosmetic compositions prepared according to the formulation example 3 and the comparative formulation examples 3 and 4 are evaluated in regards to the antibacterial ability to propionibacterium acne (ATCC 6919: medium-BHI broth) responsible for acne breakouts.

The testing method to evaluate the antibacterial ability to propionibacterium acne is as follows.

(1) Preparation of Test Bacterial Culture Medium

Propionibacterium acne is injected into the BHI broth to prepare an anaerobic culture medium.

(2) Preparation of Diluted Solution

0.15 mL of the test bacterial culture medium is added to 15 mL of BHI broth (pH 6.8) or LB broth (pH 4.5) to prepare a diluted solution.

(3) Preparation of Sample

The cosmetic compositions prepared in the formulation example 3 and the comparative formulation examples 3 and 4 are used as samples.

(4) Antibacterial Activity Testing

1) Each sample is put in the first row of a 96-well plate at the starting concentration, and the diluted solution is added at a total amount of 200 μl.

2) The mixed solutions in the wells on the first row are mixed together. 100 μl of the mixed solution is put in the wells on the second row. The mixed solutions in the wells on the second row are mixed together. 100 μl of the mixed solution is then put in the wells on the third row. In this manner, the double dilution is carried out.

3) The bacterial culture is subjected to standing culture at 32° C. for 24 hours and 48 hours, and then the degree of suspension is observed to determine the increase in the bacteria.

The minimum concentration with no increase in the bacteria is determined as the minimum inhibitory concentration (MIC). Microscopic observation is adopted in the case that the increase in the bacterium is difficult to determine.

The results of the antibacterial activity testing to propionibacterium acne are presented in Table 20. In the table, MIC is converted to the content of the active ingredient in the formulation.

TABLE 20

| Item | pH | Propionibacterium acne |
|---|---|---|
| Formulation example 3 | 5.7 | >45 ppm |
| Comparative formulation example 3 | 5.7 | Maximum concentration (no |
| | | antibacterial activity) |
| Comparative formulation example 3-1 | 5.7 | >60 ppm |
| Comparative formulation example 4 | 5.7 | >100 ppm |

The substance with the lower ppm value for MIC is more effective in regards to antibacterial activity to propionibacterium acne. The formulation example 3 leads to a considerably lower ppm value than the comparative formulation example 4 using erythromycin as a known acne treatment. This shows that the composition containing ginsenoside F2 has a far excellent antibacterial activity to the test bacterium.

### Experimental Example 16

### Test of Lipogenesis Inhibition

The mouse fibroblast cell line, 3T3-L1 cells are immobilized in a 6-well culture plate filled with Dulbecco's modified eagle's medium (DMEM) (GIBCO BRL, Life Technologies, Inc.) containing 10% fetal bovine serum (FBS) at an amount of 1×10⁵ cells/well. After 2 days, the used medium is replaced with a new DMEM (containing 10% FBS) medium to culture the cells for 2 days. Then, DMEM (containing 10% FBS) medium containing 1 μg/ml insulin, 0.5 mM IBMX, and 0.25 μM dexamethasone is used to induce the differentiation of the cultured cells, which are then treated with ginsenoside F2 and 50 μM caffeine. 2 days after the treatment, the used medium is replaced with a new DMEM medium containing insulin to culture the cells for 5 days. After 5 days, the used DMEM medium is replaced with a normal medium (DMEM containing 10% FBS). The cells are cultured and observed until they are morphologically changed into fat cells.

In order to evaluate the ginsenoside F2 in regards to the inhibitory effect on the fat accumulation in the fat cells, the completely differentiated 3T3-L1 fat cells are dyed with Sudan III (S4136, Sigma-Aldrich). The fat cells are immobilized with 4% para-formaldehyde (pH 7.2) at the room temperature in a phosphate buffer solution. The fat cells are washed with phosphate-buffered saline (PBS) and then dyed with Sudan III. The picture of the fat cells is taken for visual evaluation. The control uses a medium using neither a test substance nor a comparative substance. The comparative control group is treated with 50 μM caffeine. The fat accumulation inhibitory ability is evaluated based on the degree of dying. The degree of dying is graded +++, ++, +, and − in the decreasing order. The results are presented in Table 21.

TABLE 21

| Sample | Inhibition rate (%) |
|---|---|
| Control group (not treated) | +++ |
| Comparative group | + |
| Ginsenoside F2 | − |

As can be seen from Table 21, the ginsenoside F2 of the present invention not only leads to low-quantity fat accumulation in the fat cells but also has the higher lipogenesis inhibitory effect than caffeine which is a known lipogenesis inhibitor. Therefore, the ginsenoside F2 inhibits lipogenesis to decrease sebaceous secretions and thus suppress acne breakouts.

### Experimental Example 17

### Acne Improvement, Reduction of Sebaceous Secretion, and Skin Irritation Test

30 test objects with acne are divided into three groups, which are told to use the cosmetic compositions prepared in the formulation example 3 and the comparative formulation examples 3 and 4, respectively, for one month. Acne improvement is scored on a scale of 1 to 5 as follows.

Score 1: Not improved

Score 3: So-so

Score 5: Much improved

The experimental results are presented in Table 22, which shows the average scores of 10 patients.

The time required for acne disappearance is determined based on the number of days taken to recognize acne disappearance. And, the recurrence of acne is determined from the results obtained one month after the application. The reduction of sebaceous secretions is scored on a scale of 1 to 5 as follows.

Score 1: Not reduced

Score 3: So-so

Score 5: Much reduced

The experimental results are presented in Table 22, which shows the average scores of 10 patients. The skin irritation existence is determined as the number of test objects with skin irritations out of the total number of test objects.

TABLE 22

| | | Compara- | Compara- | Compara- |
|---|---|---|---|---|
| | | tive | tive | tive |
| | Formulation | formulation | formulation | formulation |
| | example 3 | example 3 | example 3-1 | example 4 |
| Inflammatory acne | 4.5 | 2.1 | 4.3 | 4.2 |
| improvement | | | | |
| Time required for | 2 days | 13 days | 2 days | 2 days |
| comedo acne | | | | |
| disappearance | | | | |
| Acne recurrence | NO | Yes | No | No |
| Reduction of se- | 4.3 | 2.0 | 4.2 | 4.1 |
| baceous secretion | | | | |
| Skin irritation | 0/10 | 0/10 | 0/10 | 9/10 |
| existence | | | | |

As can be seen from Table 22, compared with the comparative formulation example 3, the formulation example 3 does not lead to acne recurrence but overall has an excellent effect to improve acne. On the other hand, the comparative formulation example 4 containing a standard antibacterial substance has an effect to improve acne but causes severe skin irritations, so it is not considered as suitable for long-term use. However, the composition of the present invention causes no skin irritations and thus can be suitably used for a long time.

### Experimental Example 18

### Inflammation Improvement

1. Prostaglandin Generation Inhibitory Effect

The inflammatory effect is evaluated in terms of the inhibitory effect on prostaglandin generation. Ginsenoside F2 is used to measure the effect on macrophagocytes. Firstly, macrophagocytes collected from the abdominal cavity of a mouse are treated with aspirin to have the final concentration of 500 M, causing irreversible inhibition on the activity of cyclooxygenase (COX) remaining in the macrophagocytes. Then, 100 μl of the suspension is put in each well of a 96-well culture plate and cultured in an incubator under the conditions of 5% CO₂ and 37° C. for 2 hours to immobilize the macrophagocytes on the surface of the container. Subsequently, the immobilized macrophagocytes are washed with PBS three times and used in the inflammatory improvement test. The cultured macrophagocytes are treated with an RPMI culture medium containing 1% (w/v) LPS at the ratio of 5×10⁴ cells/ml and cultured for 12 hours to induce generation of prostaglandin. Then, the macrophagocytes are treated with 100 μl of ginsenoside F2, and the isolated prostaglandin is quantitatively analyzed by way of ELISA (Enzyme-Linked ImmunoSorbent Assey).

In this regard, the prostaglandin generation inhibitory activity of the ginsenoside F2 is determined by the reduced amount of prostaglandin generated in the LPS-treated group in comparison with the case of the control in terms of the percentage amount of the prostaglandin decreased by treatment with LPS and the sample, while the difference in the amount of the generated prostaglandin between the LPS-treated group and the control group is set to 100%. The results (prostaglandin generation inhibitory effect) are presented in Table 23.

TABLE 23

| | |
|---|---|
| Blank | 100% |
| Control (aspirin- | 25.0% |
| treated group) | |
| Ginsenoside F2 | 24.2% |

As can be seen from Table 23, the group treated with the ginsenoside F2 has a remarkable inhibitory effect on prostaglandin generation in the same manner of the control treated with aspirin.

This shows that the ginsenoside F2 of the present invention can provide an excellent effect to improve inflammation.

Further, the ginsenoside F2 is effective in inhibiting the expression of prostaglandin which is a factor causing skin inflammation, to prevent and improve skin problems.

2. IL-8 Generation Inhibitory Effect

One day before the testing, normal human skin keratinocytes (NHEK) purchased from Lonza are injected into a 96-well plate at a rate of 5×10⁴ cells/well and cultured in an incubator under conditions of 5% CO₂ and 37° C. for 24 hours. After 24 hours, the culture plate is washed with PBS twice, and the used culture medium is replaced with a serum-free keratinocyte basement medium. Ginsenoside F2 is injected into each well according to the concentration of Table 24 and allowed to react for 30 minutes. Then, the keratinocytes are treated separately with 10 μg/ml of PGSA, 50 μg/ml of PGSA, and a mixture of PGSA (50 μg/ml) and LPS (1 μg/ml). In this regard, PGSA (peptidoglycan from S. aureus) is stapylococcus-derived peptidoglycan that is a principal component of the cell membrane of gram-positive (+) bacteria, and the components of the cell membrane of bacteria are known to cause inflammation. Particularly, it is reported that about 90% of patients with atopic dermatitis are susceptible to secondary infection due to staphylococcus. LPS (lipopolysaccharide) is a principal component of the cell membrane of gram-negative (−) bacteria and reportedly a main cause of inflammation.

After the keratinocytes are cultured in an incubator under conditions of 5% CO₂ and 37° C. for 24 hours, the culture medium is collected and subjected to ELISA for interleukin-8 (IL-8). The results are presented in Table 24. The ELISA is carried out according to the testing method as instructed by the manufacturer (BD Science).

TABLE 24

| Div. | IL-8 secretion (pg/ml) |
|---|---|
| Control (not treated) | 935.12 |
| PGSA (10 μg/ml) | 4812.60 |
| PGSA (50 μg/ml) | 5895.08 |
| PGSA (50 μg/ml) + | 6814.91 |
| LPS (1 μg/ml) | |
| Ginsenoside F2 (5 ppm) | 1209.66 |
| Ginsenoside F2 (25 ppm) | 1118.80 |
| Ginsenoside F2 (50 ppm) | 1110.29 |

As can be seen from Table 24, the ginsenoside F2 considerably decreases and suppresses the secretion of IL-8 increased by PGSA and LPS. It is therefore revealed that the composition for topical skin application according to the present invention can provide an excellent anti-inflammatory effect by considerably reducing the secretion of IL-8 increased by PGSA and LPS.

### Experimental Example 19

### Evaluation of Itching Relief

One day before the testing, keratinocytes (HaCaT purchased from ATCC) are injected into a 96-well plate at a rate of 4×10⁴ cells/well and cultured in an incubator under conditions of 5% CO₂ and 37° C. for 24 hours. After 24 hours, the culture plate is washed with HBSS (Hank's Balanced Salt Solution) buffer twice, and a reaction buffer (2 μM Fluo-4-AM, 20% pluronic acid, 2.5 mM probenecid) is added to the cells. The culture plate is kept in an incubator under conditions of 5% CO₂ and 37° C. for 30 minutes and allowed to react at the room temperature for 30 minutes. Then, the culture plate is washed with HBSS buffer twice, and the cells are treated with ginsenoside F2 at a concentration (%) as given in Table 25.

After completion of the reaction for 10 minutes, the cells are treated with 2 U/ml trypsin or 5 μM PAR-2 activated peptide (SLIGKV), and then the change of Ca²⁺ concentration in the cells for 80 seconds is measured. The measurement method uses FlexStation3® (Molecular Device, U.S.A.). The difference between minimum and maximum flex values for 80 seconds after treatment with 2 U/ml trypsin or 5 μM PAR-2 activated peptide (SLIGKV) in combination with ginsenoside F2 is compared with the difference between minimum and maximum flex values for 80 seconds after treatment with 2 U/ml trypsin or 5 μM PAR-2 activated peptide (SLIGKV) alone. The inhibition rate (%) on the inflow of calcium ions into the cells is presented in Table 25.

TABLE 25

| Concentration of | Inhibition rate (%) on inflow of Ca²⁺ into cells | |
|---|---|---|
| ginsenoside F2 | Trypsin (2 U/ml) | PAR-2 activated peptide (5 μM) |
| 0.05% | 23 | 26 |
| 0.1% | 30 | 35 |
| 0.5% | 35 | 40 |
| 1.0% | 41 | 47 |

As can be seen from Table 25, the treatment with ginsenoside F2 leads to a decrease in the flow of calcium ions into the cells caused by trypsin or PAR-2 activated peptide (SLIGKV). The inflow of calcium ions into cells decreases noticeably with an increase in the concentration of the ginsenoside F2.

It is therefore revealed that the composition for topical skin application containing ginsenoside F2 according to the present invention can provide an excellent anti-itching effect by effectively inhibiting the activity of PAR-2 causing itching.

### Formulation Example 4 and Comparative Formulation Example 5

Shampoo formulations are prepared according to the compositions as given in Table 26. More specifically, a surfactant and ethyleneglycol distearate are added to purified water, and the mixture is heated up to 80° C. to homogeneously dissolve. The mixture solution is slowly cooled down to 40° C. under agitation and then mixed with the active ingredient of the present invention, a preservative, a thickener, a fragrance, and a hair conditioner. The resulting mixture is cooled down to the room temperature under agitation.

TABLE 26

| | | | Comparative |
|---|---|---|---|
| | | Comparative | formulation |
| | Formulation | formulation | example |
| Ingredient | example 4 | example 5 | 5-1 |
| Ammonium lauryl sulfate | 10 | 10 | 10 |
| ammonium polyoxyethylene | 5 | 5 | 5 |
| lauryl sulfate | | | |
| Cocoamidopropyl betaine | 2 | 2 | 2 |
| Ethylene glycol distearate | 1.5 | 1.5 | 1.5 |
| Cocoyl mono-ethanol amide | 0.8 | 0.8 | 0.8 |
| Ginsenoside F2 | 5.0 | — | — |
| Ginsenoside Rb1 | — | — | 5.0 |
| Polyquarternium-10 | 0.2 | 0.2 | 0.2 |
| Blue #1 | 0.0002 | 0.0002 | 0.0002 |
| Yellow #4 | 0.0001 | 0.0001 | 0.0001 |
| Methyl paraben | 0.1 | 0.1 | 0.1 |
| Fragrance | 0.8 | 0.8 | 0.8 |
| Citric acid | 0.1 | 0.1 | 0.1 |
| Dimethicone | 1.0 | 1.0 | 1.0 |
| Water | Up to 100 | Up to 100 | Up to 100 |

### Experimental Example 20

### Evaluation of Anti-Dandruff Ability

The formulation example 4 and the comparative formulation example 5 are measured in regards to the anti-dandruff ability and compared with each other. In this regard, the test strain is Pityrosporum Ovalae (ATCC 12078) that causes dandruff breakouts.

The measurement method for the antibacterial ability is the skin disc diffusion method (SDDM). The SDDM is a method most approaching the consumers' use behaviors to measure the antibacterial activity on the skin of guinea pig that is similar to the human skin.

More specifically, a portion of guinea pig skin is peeled off, treated with 70% ethanol, uniformly spread out and dried. The guinea pig skin is cut into a disc in a predetermined size. The sterilized skin disc is immersed in a diluted shampoo solution for 3 minutes and washed with flowing water. Then, the skin disc is placed on a solid culture medium and inoculated using the test strain to culture the test strain. The size of a clear zone with the proliferation of the test strain inhibited is measured to determine the relative antibacterial activity. The testing results are the averages of the measurement values obtained three times and presented in Table 27.

TABLE 27
Evaluation of Anti-dandruff Activity

| | Inhibited zone size (mm) | | |
|---|---|---|---|
| | | Comparative | Comparative |
| | Formulation | formulation | formulation |
| Div. | example 4 | example 5 | example 5-1 |
| Size of dandruff- | 4.7 | 0 | 2.5 |
| inhibited disc | | | |

As can be seen from Table 27, the comparative formulation example 5 not containing ginsenoside F2 does not have an effect of reducing the dandruff-causing bacteria, while the formulation example 4 containing ginsenoside F2 effectively reduces the dandruff-causing bacteria to demonstrate its excellent anti-dandruff effect.

### Experimental Example 21

### Dandruff Relief Test

24 male objects of an age of 19 to 35 years with relatively severe dandruff are divided into two groups, each group includes 12 male objects treated with the shampoo of the formulation example 4 or the comparative formulation example 5 for one month. After one month, the dandruff reduction rate is measured.

The weight of dandruff accumulated for 2 days after hair wash with a normal shampoo before the start of the testing is compared with the weight of dandruff accumulated for 2 days after completion of the testing causing the male objects to wash hair with the shampoo of the formulation example 4 or the comparative formulation example 5 every second day. In this regard, the collected dandruff is directly gathered from the scalp through a vacuum suction and used to calculate the dandruff reduction rate according the following mathematical formula 7.

The results are presented in Table 28.

$$Dandruff reduction rate ( \% ) = \frac{Dandruff weight before testing - Dandruff weight after one month of testing}{Dandruff weight ( mg ) before testing} \times 100 \qquad [ Mathematical Formula 7 ]$$

TABLE 28

| | | Comparative | Comparative |
|---|---|---|---|
| | Formulation | formulation | formulation |
| | example 4 | example 5 | example 5-1 |
| Dandruff | 44.8 | 0.7 | 23.5 |
| reduction | 56.8 | −0.5 | 36.6 |
| rate (%) | 67.2 | −1.1 | 34.9 |
| | 65.0 | 1.5 | 40.4 |
| | 46.9 | 2.2 | 39.5 |
| | 54.0 | 1.3 | 37.4 |
| | 59.3 | 6.3 | 31.2 |
| | 57.4 | 3.6 | 37.5 |
| | 65.1 | 3.3 | 36.6 |
| | 60.8 | 4.6 | 44.3 |
| | 57.9 | 3.7 | 41.2 |
| | 48.6 | 4.2 | 39.7 |
| Average | 57.0 | 2.5 | 36.9 |
| SD | 7.3 | 2.2 | 5.4 |

As can be seen from Table 28, the formulation example 4 containing ginsenoside F2 has an excellent anti-dandruff effect.

### Experimental Example 22

### Test of Scalp Anti-Itch Effect

24 male or female objects of an age of 25 to 45 years with relatively severe scalp itchiness are divided into two groups, each group includes 12 male objects treated with the shampoo of the formulation example 4 or the comparative formulation example 5 every third day for two weeks. The scalp anti-itch effect is evaluated according to the evaluation criteria as follows.

[Evaluation Criteria]

Score 5: Very excellent

Score 4: Excellent

Score 3: Good

Score 2: Poor

Score 1: Very poor

TABLE 29

| | | Comparative | Comparative |
|---|---|---|---|
| | Formulation | formulation | formulation |
| Div. | example 4 | example 5 | example 5-1 |
| Scalp anti-itch | 4.2 | 2.3 | 3.6 |
| effect | | | |

As can be seen from Table 29, the formulation example 4 containing ginsenoside F2 has an excellent effect of preventing itchiness on the scalp.

### Experimental Example 23

### Cell Proliferation Effect on Hair Follicle Dermal Papilla

The keratin protein constituting hair is produced by the keratinocytes in the hair roots, and the keratinocytes are differentiated from the hair papilla cells. To evaluate the composition of the present invention in regards to the effect to enhance the activity of hair papilla cells, the present invention uses DP6 (rat immortalized dermal papilla cell) line (Wendy Filsell, Journal of Cell Science 107, 1761-1772 (1994)). The hair papilla cells used in the present invention are a cell line isolated from the hair roots of male PVG rats by the microdissection method and cultured with DMEM (Dulbecco's modified eagle's medium, Gibco BRL, Gaithersburg, Md., U.S.A.) containing 10% FBS (Fetal Bovine Serum) in an incubator under the conditions of 5% CO₂ and 37° C. for 24 hours. DP6 is put in the 96-well plate, cultured in an incubator at 37° C. for 24 hours and treated with the ginsenoside F2 of the present invention independently at a concentration of 5 ppm, 10 ppm, or 20 ppm. 24 hours after the treatment, the cell proliferation ability is measured using a WST-1 kit (Roche). The results are presented in Table 30.

TABLE 30

| Div. | Cell proliferation ability (%) |
|---|---|
| Control (not treated) | 100 |
| Ginsenoside Rb1 (5 ppm) | 114 |
| Ginsenoside Rb1 (10 ppm) | 118 |
| Ginsenoside Rb1 (20 ppm) | 127 |
| Ginsenoside F2 (5 ppm) | 116 |
| Ginsenoside F2 (10 ppm) | 122 |
| Ginsenoside F2 (20 ppm) | 138 |

As can be seen from Table 30, the treatment with the ginsenoside F2 leads to an increase in the proliferation of hair papilla cells, which increases on a concentration-dependent basis with significant difference.

### Experimental Example 24

### Evaluation of Effect of Increasing Activity of Calcium Ion Channel

Minoxidil, a treatment for hair loss, is known as a potassium ion channel opener (KATP channel opener) potentially acting on mitochondria and a representative drug used for treatment of androgenic alopecia (hair loss). A testing method used to evaluate the action of minoxidil includes treating with tolbutamide (SIGMA-ALDRICH, T0891) blocking the potassium ion channels (KATP channels) in fibroblasts constituting the corium of scalp to inhibit cell proliferation and then opening the potassium ion channels (KATP channels) to resume the cell proliferation.

In order to evaluate the function of the composition of the present invention as a KATP channel opener, the present invention uses a mouse embryonic fibroblast cell line, NIH3T3, as fibroblasts. The cell line is obtained by naturally immortalizing a fibroblast cell line isolated from an NIH Swiss mouse embryo with 3T3 protocol. The cell line is cultured with DMEM (Dulbecco's modified eagle's medium, Gibco BRL, Gaithersburg, Md., U.S.A.) containing 10% FBS (Fetal Bovine Serum) in an incubator under the conditions of 5% CO₂ and 37° C. for 24 hours. NIH3T3 is put in the 96-well plate, cultured in an incubator at 37° C. for 24 hours and treated with 2.5 mM tolbutamide. 10 minutes later, NIH3T3 is treated with 10 μM minoxidil or ginsenoside F2 independently at a concentration of 2.5 ppm, 5 ppm, or 10 ppm. 48 hours after the treatment, the cell proliferation ability is measured using a WST-1 kit (Roche). The results are presented in Table 31.

TABLE 31

| Div. | Cell proliferation ability (%) |
|---|---|
| Control (not treated) | 100 |
| Minoxidil | 132 |
| Ginsenoside Rb1 (2.5 ppm) | 113 |
| Ginsenoside Rb1 (5 ppm) | 117 |
| Ginsenoside Rb1 (10 ppm) | 124 |
| Ginsenoside F2 (2.5 ppm) | 115 |
| Ginsenoside F2 (5 ppm) | 120 |
| Ginsenoside F2 (10 ppm) | 131 |

As can be seen from Table 31, the treatment with the ginsenoside F2 recovers the proliferation of fibroblasts, which increases on a concentration-dependent basis with significant difference. It is also revealed that the treatment with 10 ppm of ginsenoside F2 can recover the cell proliferation at an equivalent level of the treatment with minoxidil.

### Formulation Example 5 and Comparative Formulation Example 6

The composition of a cream base is prepared by a typical method according to the composition of Table 32 (unit: wt. %).

TABLE 32

| | | Comparative | Comparative |
|---|---|---|---|
| | Formulation | formulation | formulation |
| Ingredient | example 5 | example 6 | example 6-1 |
| Labrafac | 23 | 23 | 23 |
| Tween 80 | 10.5 | 10.5 | 10.5 |
| Glycerol mono- | 4.0 | 4.0 | 4.0 |
| stearate | | | |
| Stearic acid | 7.0 | 7.0 | 7.0 |
| Cetyl alcohol | 2.0 | 2.0 | 2.0 |
| Propylene glycol | 9.0 | 9.0 | 9.0 |
| Glycerol mono- | 3.5 | 3.5 | 3.5 |
| oleate | | | |
| Ginsenoside F2 | 1 | — | — |
| Ginsenoside Rb1 | — | — | 1 |
| Water | 40 | 41 | 40 |

### Experimental Example 25

### Effect of Ginsenoside F2 to Stimulate Hair Growth

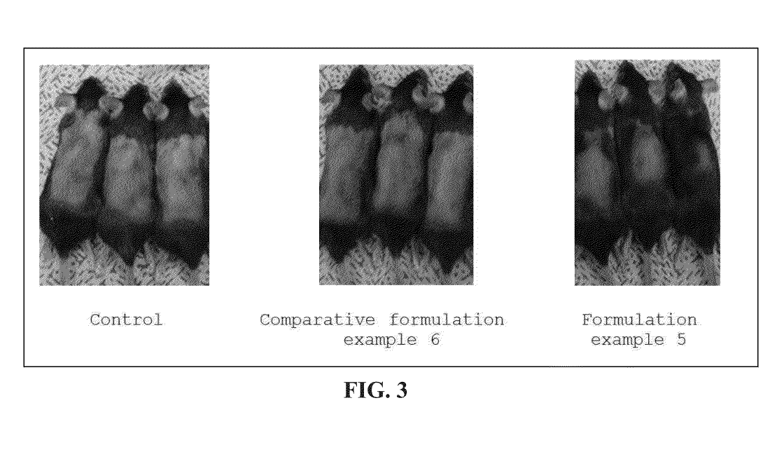

An ICR mouse is depilated on the back side and completely removed of hair using a shaving cream (Veet cream). For two test groups other than the control (normal group), 200 μl of 1% dinitrochlorobenzene (DNCB) is applied to the skin once a day to induce inflammation on the skin for 3 days. 3 days later, the cream base of the formulation example 5 or the comparative formulation example 6 is applied to the skin of the groups other than the control group once a day. The hair growth in each group is observed. The results are presented in FIG. 3.

Referring to FIG. 3, the control group does not show any sign of hair growth in the shaved region of the skin during the 15 days of observation. The group treated with the cream base not containing ginsenoside F2 gets a slight hair growth effect, and the group treated with the cream base containing ginsenoside F2 has remarkable hair growth overall the shaved region of the skin.

### Experimental Example 26

### Hair Nourishing Effect of Ginsenoside F2

The procedures are performed in the same manner as described in the experimental example 25 to treat the mouse shaved on the back with dinitrochlorobenzene (DNCB) causing inflammation on the skin and ultimately deteriorate the hair growth by providing stress conditions in the skin. To evaluate the hair regeneration efficacy of the composition of the present invention, the creams of the formulation example 5 containing ginsenoside F2 and the comparative formulation example 6 are independently used. Then, the length of hair grown as a function of the time period of treatment is measured in comparison with the case of the control group. The results are presented in Table 33.

TABLE 33

| | Hair length (cm) | | |
|---|---|---|---|
| | | Treatment with | Treatment with |
| Period of | Treatment with | comparative | comparative |
| treatment | formulation | formulation | formulation |
| (days) | example 5 | example 6 | example 6-1 |
| 0 | 0.1 | 0.1 | 0.1 |
| 6 | 0.2 | 0.1 | 0.2 |
| 9 | 0.4 | 0.1 | 0.3 |
| 12 | 1.1 | 0.1 | 0.6 |

Referring to Table 33, the group treated with the cream base of the formulation example 5 has the length of hair with a statistically significant difference (p<0.01) with respect to the group treated with the cream base of the comparative formulation example 6. Further, the group treated with the cream base of the formulation example 5 has the length of hair increased with an increase in the number of days under treatment (for example, the hair is grown up to about 1.1 cm in the 12^{th} day of treatment), ending up with longer hair than the group treated with the cream base of the comparative formulation example 6 not containing ginsenoside F2.

This shows that the ginsenoside F2 promotes hair regeneration under the hair regeneration inhibitory conditions formed due to the stress imposed on the skin and hence has a hair regeneration function for a loss of hair caused under stress.

### Experimental Example 27

### Melanogenesis Promoting Effect of Ginsenoside F2

Melanin-a cells are injected in a 24-well microtiter plate at a rate of 50,000 cells per well with an RPMI medium containing 5% fetal bovine serum, 100 IU penicillin G, and 0.2 μM TPA. Next day, the cells are treated with ginsenoside F2 as a test substance independently at the final concentration of 10 ppm or 50 ppm. The negative control is treated with 0.1% DMSO, and the positive control is treated with 100 μM IBMX. Then, the cells are cultured at 37° C. for 3 days. The wells of the plate are washed with PBS, and 100 μl of 1N NaOH is added to each well to dissolve melanin in the cells. The absorbance of the dissolved melanin is measured at 405 nm with a microplate reader. The effect of the ginsenoside F2 to promote melanogenesis is evaluated in comparison with that of the control groups. The results are presented in Table 34.

TABLE 34

| Sample | Yield of Melanin (%) |
|---|---|
| DMSO (0.1%) | 100 |
| IBMX (100 μM) | 120 |
| Ginsenoside Rb1 (10 ppm) | 112 |
| Ginsenoside Rb1 (50 ppm) | 120 |
| Ginsenoside F2 (10 ppm) | 113 |
| Ginsenoside F2 (50 ppm) | 129 |

Referring to Table 34, the ginsenoside F2 promotes the melanogenesis of melanocytes to increase the yield of melanin and thus has an excellent melanogenesis promoting effect.

### Experimental Example 28

### Effect of Ginsenoside F2 to Promote Expression of MITF and Tyrosinase in Melanocytes

501me1 cell line is injected in each well of a 6-well microtiter plate at a rate of 500,000 cells/well. The cell line in each well is treated with 0.1% of DMSO as the negative control group, 100 μM of IBMX as the positive control group, or ginsenoside 10 ppm of F2 as the test group, and cultured at 37° C. for 24 hours, 48 hours, or 72 hours, to obtain proteins. The proteins thus obtained are subjected to Western Blot using MITF and tyrosinase antibodies. Protein extraction and Western Blot are performed using the standard methods known in the art. The results of the Western Blot are calculated in comparison with 100 of the negative control group and presented in Table 35.

TABLE 35

| | MITF | | | Tyrosinase | | |
|---|---|---|---|---|---|---|
| | | Ginsen- | Ginsen- | | Ginsen- | Ginsen- |
| | | oside | oside | | oside | oside |
| | IBMX | F2 | Rb1 | IBMX | F2 | Rb1 |
| 24 hrs | 121 | 92 | 93 | 160 | 150 | 149 |
| 48 hrs | 98 | 120 | 116 | 149 | 182 | 165 |
| 72 hrs | 224 | 162 | 144 | 298 | 211 | 189 |

Referring to Table 35, the ginsenoside F2 increases the expression of MITF and tyrosinase proteins in the melanocytes.

### Experimental Example 29

### Evaluation of Ginsenoside F2 in Regards to Effect of Preventing Grey Hair and Stimulating Black Hair Growth Using Mouse with Vitiligo and Lots of Grey Hair

A mouse with vitiligo (C57b1/6-Mitf^{mi-vit}) is purchased from The Jackson Lab in U.S.A. The testing method on the grey hair inhibitory effect using a mouse with lots of grey hair is carried out as follows. A 12-week old mouse is subjected to depilation on the back side. The area of the depilated region is controlled to be the same to every object. From the next day of the depilation, the grey hair inhibitory substances are applied on the depilated region twice a day. The vehicle for the grey hair inhibitory substances is the mixture of EtOH, 1,3-BG and DW (3:2:5 in volume ratio). The liquid containing the vehicle alone is the negative control, the liquid containing the vehicle in combination with 50 mM IBMX is the positive control, and the liquid containing the vehicle in combination with 2.5% ginsenoside F2 is the test group. When the difference in the grey hair inhibitory effect between the substances is visually recognizable in about 3 weeks, the newly grown hair is collected to measure the quantity of melanin produced in the hair. The quantity of melanin in the hair is measured using esperase (Novozyme) strains that are a protein hydrolase. Esperase is dissolved in a buffer solution (50 mM tris-HCl, 5 mM DTT, pH 9.3) to a concentration of 1 NPU/ml to prepare a reaction buffer. 5 mg of mouse hair is put in 1 ml of the reaction buffer, which is stirred at a speed of 1,000 rpm at 37° C. for 13 hours to cause the reaction and then subjected to a momentary centrifugal isolation to separate the reactant solution from hair. The reactant solution thus obtained is placed in each well of a 96-well plate, and the absorbance at 405 nm is measured to determine the amount of melanin in the reactant solution. When a mouse with vitiligo and lots of grey hair is treated with the negative control substance, the positive control substance, or the test substance, the efficacies are visually observed, and the melanin in hair is quantitatively analyzed. The results are presented in Table 36.

TABLE 36

| | Negative | | Ginsenoside | Ginsenoside |
|---|---|---|---|---|
| | control | IMBX | F2 | Rb1 |
| Amount of melanin | 100 | 105.9 | 110.8 | 107.1 |
| (%) with respect to | | | | |
| control | | | | |

Referring to Table 36, the ginsenoside F2 inhibits the growth of grey hair in the mouse with lots of grey hair and increases the amount of melanin in hair to promote the occurrence of black hair.

### Experimental Example 30

### Evaluation on Antibacterial Effect of Ginsenoside F2

The antibacterial testing is carried out in order to evaluate the antibacterial effect of ginsenoside F2. The detailed testing method is as follows.

Staphylococcus aureus, Escherichia coli, and pseudomonas aeruginosa strains are cultured with a tryptic soy broth, and candida albicans and aspergillus niger strains are cultured with sabouraud dextrose broth. The test bacterial liquid is the 1/100 (1/10 for candida albicans strain) diluted solution of the culture medium diluted with each medium. For aspergillus niger, the test bacterial liquid is the spore suspension prepared to 2×10⁸ cfu/ml.

0.15 ml of the test bacterial liquid is added to 15 ml of each culture medium to prepare a well-blended diluted solution.

16 μl of the sample is put into each well on the first row of a 96-well plate, and 184 μl of the diluted solution is added to each well on the first row. Then, 100 μl of the diluted solution is put into each of the other wells. The mixed solution of the first row is sufficiently stirred, and 100 μl of the mixed solution is added to each well on the second row of the 96-well plate and stirred sufficiently. 100 μl of the mixed solution on the second row is taken and added to each well on the third row to achieve two-fold serial dilution.

Straphylococcus aureus, Escherichia coli, and Pseudomonas aeruginosa strains are cultured in a thermostat container at 32°, and Candida albicans and Aspergillus niger strains are cultured in a thermostat container at 25° C.

48 hours later, the cell proliferation is determined by the turbidity index and microscopic observation to determine the minimum inhibitory concentration (MIC). The results are presented in Table 37.

TABLE 37

| Sample | | Ginsenoside F2 |
|---|---|---|
| MIC (%) | Pseudomonas aeruginosa | 0.09 |
| | Straphylococcus aureus | 0.07 |
| | Escherichia coli | 0.13 |
| | Candida albicans | >4 |
| | Aspergillus niger | >2 |

As can be seen from Table 37, the ginsenoside F2 has an antibacterial ability on various bacteria. This implicitly shows that the ginsenoside F2 acts as a natural preservative or an antibacterial agent in the composition.

What is claimed is:

1. A method of treatment selected from the group consisting of providing an enhancing skin elasticity, improving skin wrinkles, whitening the skin, providing a moisturizing effect to the skin, enhancing a skin barrier function, inducing differentiation of keratinocytes on the skin, improving acne, providing an antibacterial effect, providing an anti-inflammatory effect, inhibiting lipogenesis, improving atopy, improving skin complexion and skin tone, tightening skin pores, controlling sebaceous secretion, improving skin problems, providing an anti-dandruff effect, and enhancing or stimulating hair growth,
the method comprising topically applying to skin of a subject in need of the treatment an effective amount of a composition comprising, as an active ingredient, ginsenoside F2 represented by the following chemical formula 1 and extracted from roots and leaves of clean fresh ginseng cultivated in an aggregate hydroponic ginseng cultivation system or an aeroponic ginseng cultivation system:

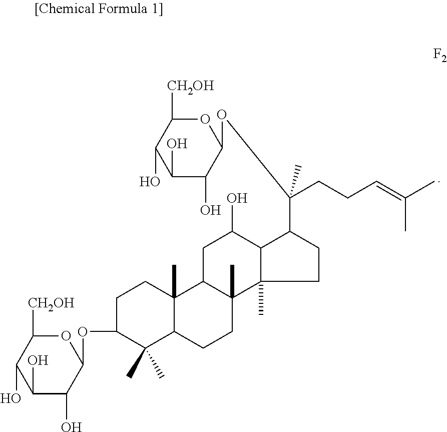

2. The method of treatment as claimed in claim 1, wherein the roots and leaves of clean fresh ginseng are produced by a method using an aggregate hydroponic ginseng cultivation system comprising:
a) performing a first acclimatization step of storing a ginseng seedling in a storage greenhouse at 15° C. for one or two days after a release and then tentatively planting the ginseng seedling;
b) performing a second acclimatization step of storing the tentatively planted ginseng seedling in a greenhouse for one or two days to have the ginseng seedling acclimatized in the environment of the greenhouse and then finally planting the ginseng seedling in a mixed medium formed in a bed with a drainage groove;
c) preparing a nutrient solution;
d) supplying an appropriate amount of the nutrient solution for the ginseng seedling; and
e) harvesting after 4 to 5 months.

3. The method of treatment as claimed in claim 1, wherein the roots and leaves of clean fresh ginseng are produced by a method using an aeroponic ginseng cultivation system comprising:
a) performing a first acclimatization step of storing a ginseng seedling in a storage greenhouse at 15° C. for one or two days after a release and then tentatively planting the ginseng seedling;
b) performing a second acclimatization step of storing the tentatively planted ginseng seedling in a greenhouse for one or two days to have the ginseng seedling acclimatized in the environment of the greenhouse and then finally planting the ginseng seedling in a bed;
c) preparing a nutrient solution;
d) spraying the nutrient solution on a root of the ginseng seedling through a mist nozzle;
e) recycling the used nutrient solution transferred to a nutrient solution tank through a drainage opening formed on the one end of the bed; and
f) harvesting after 4 to 5 months.

4. The method of treatment as claimed in claim 1, wherein the ginsenoside F2 is contained at an amount of 0.001 to 50 wt. % with respect to the total weight of the composition.
